(19) United States
(12) Patent Application Publication
(10) Pub. No.: US 2010/0204076 A1
(43) Pub. Date: Aug. 12, 2010

(54) GELLED NAIL POLISH REMOVER

(76) Inventor: WEI-YUN CHENG, TAOYUAN COUNTY 32543 (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/703,845

(22) Filed: Feb. 11, 2010

(30) Foreign Application Priority Data
Feb. 12, 2009 (TW) 098141103

(51) Int. Cl. A61K 8/04; A61Q 3/04

(52) U.S. Cl. 510/118

(57) ABSTRACT

A gelled nail polish remover includes of a thickening agent of approximately 1.0%-5.0% by weight, low volatility organic solvents of approximately 85%-99% by weight, a skin conditioning agent of approximately 0.1%-5.0% by weight, and an antioxidant of approximate 0.15%-0.3% by weight, which are mixed to form a gel for removing nail polish and featuring low toxin, low volatility, low flammability, and almost no odor smell.

### BACKGROUND OF THE INVENTION

1. Field of the Invention

The present invention relates to a gel containing organic solvent of low volatility, and in particular to a gelled nail polish remover for conveniently removing nail polish and having characteristics of low toxin, low volatility, low flammability, and low odor, making the gelled nail polish meet regulatory requirements for VOC (Volatile Organic Compound) standard.

2. The Related Arts

Conventionally, a nail polish remover contains free-flowing liquid solvents, such as acetone, ethyl acetate, and methyl ethyl ketone. These solvents have undesired properties, such as volatility, flammability, and strong odor, making them not meet the current requirement for environmental protection.

To overcome the above mentioned drawbacks of the solvents that were conventionally used in nail polish removers, some modern nail polish removers are formulated to contain new solvents that feature low volatility, high flash point, and low odor, such as dibasic ester, propylene carbonate, diethylene glycol monobutyl ether. These solvent satisfy the current trend of environmental protection and work excellent as nail polish removers. However, these solvents are free-flowing liquids that suffer easy spillage and overflowing, making it inconvenient for users to carry and use.

Formulas of cream like solvents were available from prior art patent documents for improving the above discussed problems. However, those formulas of cream like solvent contain toxicant substances that either do not meet VOC related regulations or is harmful to human bodies. For example, U.S. Pat. No. 4,804,486, which is titled “Nail Polish Remover”, discloses a gel like solvent that contains a solvent of dihydro-2(3H)-furanone (GBL) (CAS No. 96-48-0) that is a subject of an FDA Talk Paper in 1999 as a harmful substance.

Further, U.S. Pat. No. 5,543,085, titled “Cleaning Apparatus and Method for Fuel and Other Passages”, discloses a thickened nail polish remover that consists of a volatile solvent, ethyl acetate, which has a vapor pressure more than 0.1 mmHg at 20° C. (68° F.) and des not meet the VOC standard.

U.S. Pat. No. 6,071,865, tilted “Nail Polish Remover”, discloses a gel nail polish remover containing N-methyl-2-pyrrolidone (NMP) and gamma-butyrolactone (GBL), both solvents being considered harmful to human body heath.

### SUMMARY OF THE INVENTION

The conventional nail polish removing solvents or nail polish removing gels contain organic solvent compositions that are volatile, toxicant, and odorant. This makes the conventional nail polish removing solvents or gels not meet the VOC related regulations for environmental protection. These substances are also harmful to human bodies and this makes them less valuable in the industry.

Thus, an objective of the present invention is to provide a gelled nail polish remover composition comprising:

a gelling agent from about 1.0% to about 5% by weight, the gelling agent being selected from a group consisting of hydroxypropyl cellulose ((C₃₆H₄₄O₁₈)ₙ), cross-linked acrylic acid polymer (C₂H₁₀O₂), and acrylate/C₁₀₋₃₀ alkyl acrylate cross-polymer;

a solvent from about 85% to about 99% by weight, the solvent including dibasic ester and glycol ether; and

a skin conditioning agent from about 0.1% to about 5.0% by weight and an antioxidant from about 0.15% to about 0.3% by weight.

In the gelled nail polish remover in accordance with the present invention, the gelling agent of hydroxypropyl cellulose has a molecular weight in a range from about 850,000 to about 1,150,000.

In the gelled nail polish remover in accordance with the present invention, the dibasic ester used in the solvent is a mixture of three dibasic esters including dimethyl succinate (CH₃OOC(CH₂)₂COOCH₃), dimethyl glutarate (CH₃OOC(CH₂)₃COOCH₃), and dimethyl adipate (CH₃OOC(CH₂)₄COOCH₃), taking a ratio from about 0% to about 70%.

In the gelled nail polish remover in accordance with the present invention, the glycol ether used in the solvent is diethylene glycol monobutyl ether (C₄H₉(OCH₂CH₂)₂OH), taking a ratio from about 25% to about 99%.

The gelled nail polish remover in accordance with the present invention contain a solvent that is free of volatile organic substances that are prohibited by VOC related regulations and features low toxin, low volatility, low flammability, and low odor, making the gel of the present invention meet the requirement of environmental protection and not harmful to human bodies. The gel of the present invention can be packaged in a twist-up plastic container or a flexible plastic tube whereby crash or spillage can be eliminated.

The gelled nail polish remover of the present invention completely overcomes the drawbacks of high volatility, high flammability, high toxin, inconvenience of use and strong odor that are found the conventional nail polish removers and also satisfy the current trend of environmental protection and enhance industrial value thereof.

### DETAILED DESCRIPTION OF THE PREFERRED EMBODIMENTS

The present invention provides a gelled nail polish remover of which a preferred embodiment will be described to facilitate understanding of the present invention. The gelled nail polish remover in accordance with the present invention has a composition comprising the following constituents:

A gelling agent component takes a percentage of approximately 1.0%-50% by weight and contains one to three components selected from hydroxypropyl cellulose (HPC), such as that available from BASF under trade name KLUCEL HPC (CAS No. 9004-64-2), cross-linked acrylic acid polymer (C₂H₁₀O₂) or Carbomer, such as that available from Lubrizol Advanced Materials Inc. under trade name Carbopol Ultrez-10 (CAS No. 195739-91-4), and acrylates/C₁₀₋₃₀ alkyl acrylate cross-polymer, such as that available from Lubrizol Advanced Materials Inc. under trade name Carbopol Ultrez-20 and Carbopol Ultrez-21.

All of above mentioned thickeners are useful for gelling agent. The usage is from 1.5% to 5.0% and preferred percentages are 1.4% for hydroxypropyl cellulose, or 2.5% for cross-linked acrylic acid polymer or 2.5% for acrylates/C₁₀₋₃₀ alkyl acrylate cross-polymer.

A solvent takes a percentage of approximately 85%-99% by weight. The solvent comprises dibasic ester (DBE) and diethylene glycol monobutyl ether. Both of the solvents exempt from the VOC content limit, having a vapor pressure less than 0.1 mm Hg at 20° C.

The dibasic ester used in the solvent includes a mixture of dimethyl succinate (CH₃OOC(CH₂)₂COOCH₃) (CAS No. 106-65-0), dimethyl glutarate (CH₃OOC(CH₂)₃COOCH₃) (CAS No. 1119-40-0), and dimethyl adipate (CH₃OOC(CH₂)₄COOCH₃) (CAS No. 627-93-0), which is a non-toxicant colorless transparent liquid having slight fragrance and is an environment-conservative high boiling point solvent that is biodegradable. A referred mixture ratio of these dibasic esters is approximately 15-25% dimethyl succinate (CH₃OOC(CH₂)₂COOCH₃), approximately 55-60% dimethyl glutarate (CH₃OOC(CH₂)₃COOCH₃), and approximately 10-25% dimethyl adipate (CH₃OOC(CH₂)₄COOCH₃).

Preferably, the dibasic ester contained in the solvent takes a percentage of approximately 0%-70% by weight of the gelled nail polish remover of the present invention. For example, in case hydroxypropyl cellulose is used as the thickener, the preferred percentage is approximately 60%, while when cross-linked acrylic acid polymer or acrylate/C₁₀₋₃₀ alkyl acrylate cross-polymer is used as the thickener, the preferred percentage is approximately 20%.

Diethylene glycol monobutyl ether (C₄H₉(OCH₂CH₂)₂OH) contained in the solvent mentioned above is a solvent featuring low toxin, low flammability, and low volatility and having a saturation vapor pressure less than 0.1 mmHg at 20° C.(68° F.) and providing excellent result of removing nail polish. To make a gelled solvent, the thickener must be soluble in solvents and the diethylene glycol monobutyl ether shows excellent solubility for the above mentioned thickeners. A gelled solvent having a viscosity greater than 35,000 cps can be obtained by mixing at least 30% diethylene glycol monobutyl ether and at most 68% dibasic ester.

A mixture of at least 70% diethylene glycol monobutyl ether and at most 28% dibasic ester of the solvent and 25% the thickener of cross-linked acrylic acid polymer or acrylate/C₁₀₋₃₀ alkyl acrylate cross-polymer provides a gelled solvent having a viscosity greater than 25,000 cps.

Water and neutralizing agent are not necessary for making the above gelled solvent. Diethylene glycol monobutyl ether contained in the solvent according to the present invention takes a percentage of approximately 25%-99% by weight. When hydroxypropyl cellulose is used as the thickener, the preferred percentage is approximately 35%, while when cross-linked acrylic acid polymer or acrylate/C₁₀₋₃₀ alkyl acrylate cross-polymer is used as the thickener, the preferred percentage is approximately 75%.

A skin conditioning and antioxidant agent is also included, wherein the skin conditioning component takes a percentage of use of approximately 0.1%-5.0% and the antioxidant takes a percentage of approximately 0.15%-0.3%. The skin conditioning component is selected from a group including lanolin, vitamin E, acetic ester, panthenol and aloe vera. The antioxidant is selected from a group including methylparaben (C₈H₈O₃) (CAS No. 99-76-3) and propylparaben (C1011₁₂O₃) (CAS No. 94-13-3).

Several examples of the gelled nail polish removers according to the present invention will be given.

Example 1 of the gelled nail polish remover comprises the following ingredients, which are expressed in percentage (%) by weight: diethylene glycol monobutyl ether 35.5%, dibasic ester 58.0%, hydroxypropyl cellulose 1.5%, propylene glycol 3.8%, lanolin 1.0%, methylparaben 0.1%, and propylparaben 0.1%.

Example 2 of the gelled nail polish remover comprises the following ingredients, which are expressed in percentage (%) by weight: diethylene glycol monobutyl ether 74.0%, dibasic ester 20.0%, cross-linked acrylic acid polymer 2.7%, propylene glycol 3.0%, panthenol 0.1%, methylparaben 0.1%, and propylparaben 0.1%.

The present invention provides a gelled nail polish remover with many advantages including easy application in an easily measured amount, conservation of product and safety for user, reduction of impact to the natural environment, simple and easy production, and lower production cost for manufacture.

Although the present invention has been described with reference to the preferred embodiments and examples thereof, it is apparent to those skilled in the art that a variety of modifications and changes may be made without departing from the scope of the present invention which is intended to be defined by the appended claims.

What is claimed is:

1. A gelled nail polish remover, comprising:
a gelling agent having a percentage of approximately 1.0%-5.0% by weight, the gelling agent being selected from hydroxypropyl cellulose ((C₃₆H₄₄O₁₈)ₙ), cross-linked acrylic acid polymer (C₂H₁₀O₂), and acrylate/C₁₀₋₃₀ alkyl acrylate cross-polymer;
a solvent having a percentage of approximately 85%-99% by weight, the solvent comprising dibasic ester and glycol ether; and
a skin conditioning agent having a percentage of approximately 0.1%-5.0% by weight and an antioxidant having a percentage of approximately 0.15%-0.3% by weight.

2. The gelled nail polish remover as claimed in claim 1, wherein hydroxypropyl cellulose contained in the gelling agent has a molecular weight in a range of approximately 850,000-1,150,000.

3. The gelled nail polish remover as claimed in claim 1, wherein the dibasic ester contained in the solvent is a mixture of three dibasic esters including dimethyl succinate (CH₃OOC(CH₂)₂COOCH₃), dimethyl glutarate (CH₃OOC(CH₂)₃COOCH₃), and dimethyl adipate (CH₃OOC(CH₂)₄COOCH₃).

4. The gelled nail polish remover as claimed in claim 3, wherein the dibasic ester takes a percentage of approximately 0%-70%.

5. The gelled nail polish remover as claimed in claim 1, wherein the glycol ether contained in the solvent is diethylene glycol monobutyl ether (C₄H₉(OCH₂CH₂)₂OH).

6. The gelled nail polish remover as claimed in 5, wherein diethylene glycol monobutyl ether (C₄H₉(OCH₂CH₂)₂OH) takes a percentage of approximately 25%-99%.

7. The gelled nail polish remover as claimed in 1, wherein the skin conditioning agent is selected from a group including lanolin, vitamin E, acetic ester, panthenol and aloe vera.

8. The gelled nail polish remover as claimed in 1, wherein the antioxidant is selected from methylparaben (C₈H₈O₃) and propylparaben (C₁₀H₁₂O₃).
